(19) United States
(12) Patent Application Publication
(10) Pub. No.: US 2009/0216104 A1
(43) Pub. Date: Aug. 27, 2009

(54) USE OF ACID DERIVATIVES OF FLUOROPOLYMERS FOR FOULING-RESISTANT SURFACES

(76) Inventors: Joseph M. DeSimone, Chapel Hill, NC (US); Michael S. Williams, Santa Rosa, CA (US); Lauren Portnow, Chapel Hill, NC (US); Colin Wood, Carrboro, NC (US); Zhilian Zhou, Chapel Hill, NC (US); Everett Baucom, Chapel Hill, NC (US); Ginger Denison Rothrock, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/064,273

(22) PCT Filed: Aug. 28, 2006
(86) PCT No.: PCT/US06/33699

§ 371 Date: Sep. 18, 2008

Related U.S. Application Data
(60) Provisional application No. 60/711,493, filed on Aug. 26, 2005.

(51) Int. Cl. A61L 27/28; B05D 3/02; A61B 17/58; A61L 17/00; A61N 1/02; A61F 2/16; A61B 5/00; A61B 17/32; A61L 29/14; A61B 17/3201; A61M 5/00

(52) U.S. Cl. 600/365; 427/385.5; 427/508; 427/2.24; 427/2.25; 427/2.12; 427/2.26; 427/2.13; 427/388.1; 606/76; 606/231; 623/1.46; 623/1.42; 607/2; 607/5; 607/9; 623/6.62; 607/57; 606/167; 604/523; 606/174; 604/187

(57) ABSTRACT

The presently disclosed subject matter describes acid-derivatized perfluoropolyether (PFPE) materials and their use as coatings, sealants, and flexible fillers for devices, apparatuses, and structural parts for a variety of medical applications, and as coatings, sealants, flexible fillers, and structural parts for vessels, structures, and machinery exposed to a marine environment.

### CROSS-REFERENCE TO RELATED APPLICATION

This application claims the benefit of U.S. Provisional Application No. 60/711,493, filed Aug. 26, 2005, the disclosure of which is incorporated by reference herein in its entirety.

### GOVERNMENT INTEREST

A portion of the disclosure contained herein was made with U.S. Government support from the Office of Naval Research Grant No. N00014-02-1-0185. The U.S. Government has certain rights to that portion of the disclosure.

### TECHNICAL FIELD

The presently disclosed subject matter generally relates to acid-derivatized perfluoropolyethers and their use in providing fouling-resistant surfaces for a variety of substrates, including, but not limited to, implantable or insertable medical devices, marine vessels and structures, and the like.

### ABBREVIATIONS

AETMAC=acryloxyethyltrimethylammonium chloride
AV=arterio-venous
° C.=degrees Celsius
cm=centimeter
DBTDA=dibutyltin diacetate
DMA=dimethylacrylate
DMPA=2,2-dimethoxy-2-phenylacetophenone
DNA=deoxyribonucleic acid
EIM=2-isocyanatoethyl methacrylate
FEP=fluorinated ethylene propylene
FGF=fibroblast growth factor
Freon 113=1,1,2-trichlorotrifluoroethane
g=grams
h=hours
HCl=hydrochloric acid
HEA=hydroxyethylacrylate
Hz=hertz
IR=infra red
kg=kilograms
kHz=kilohertz
KOH=potassium hydroxide
kPa=kilopascal
mg=milligram
MgSO₄=magnesium sulfate
MHz=megahertz
mL=milliliters
mm=millimeters
mmol=millimoles
mN=milli-Newton
m.p.=melting point
MRI=magnetic resonance imaging
mW=milliwatts
NaOH=sodium hydroxide
nm=nanometers
PDGF=Platelet-Derived Growth Factor
PDMS=polydimethylsiloxane
PEG=poly(ethylene glycol)
PEGdiA=poly(ethylene glycol)diacrylate
PFPE=perfluoropolyether
PLA=poly(lactic acid)
psi=pounds per square inch
PVDF=poly(vinylidene fluoride)
PTFE=polytetrafluoroethylene
S-PFPE=styrene-capped perfluoropolyether
TBT=tri-n-butyl tin
Tg=glass transition temperature
Tm=crystalline melting temperature
TMPTA=trimethylolpropane triacrylate
μm=micrometers
UV=ultraviolet
W=watts
wt=weight
ZDOL=poly(tetrafluoroethylene oxide-co-difluoromethylene oxide)α,ω diol

### BACKGROUND

The fouling of a surface, such as that of an implanted medical device or a marine vessel or marine structure, can diminish the efficiency and functionality of that surface. One cause of such fouling in both implanted medical devices and marine vessels or marine structures is the adsorption of proteins on the surfaces thereof. For example, the extracellular matrix proteins and blood borne proteins of biological media can cause biofouling of implanted medical devices. Likewise, proteinaceous material from marine organisms can cause biofouling of marine vessels and/or marine structures.

Biofouling can be a major contributor to the failure of in vivo biosensors and other implanted biomedical devices. Blood borne proteins and platelets can begin to adhere to the sensor surface from minutes to hours after the implantation of a medical device. After a few days, the immune system can begin to react, and proteins and cells begin to adhere, thereby causing inflammation. Tissue formation and vascularization can begin as the body attempts to repair the wound. Within weeks or months, encapsulation can occur, in which the avascularity and fibrocity of the tissue can increase. All of these responses can contribute to potential patient infections, compromised device performance, or even device failure.

One approach to control the inflammation and fibrosis resulting from tissue trauma at the site of implantation has been to use inert materials, such as titanium or single-crystalline alumina, as disclosed in U.S. Pat. No. 4,122,605 to Hirabavashi et al., to fabricate the implanted medical device. While suitable for bone or tooth implants, such materials are not useful in more complex prosthetic devices or in biosensors. Another approach has been the use of a porous, outer coating of a polyester (e.g., DACRON®, INVISTA, Wilmington, Del., United States of America) or a polytetrafluoroethylene, (e.g., TEFLON®, E.I. duPont de Nemours and Co., Wilmington, Del., United States of America) as disclosed in U.S. Pat. No. 4,648,880 to Brauman et al., or with polytetrafluoroethylene (PTFE), as disclosed in U.S. Pat. No. 5,779,734 to Ledergerber. Such coatings, however, are not practical for prosthetic devices or biosensors having complex geometries. For example, it is difficult to coat PTFE onto small medical devices with precision while maintaining the device features because PTFE typically requires high temperature melt processing steps and is not soluble in any solvent at temperatures suitable for use in coating medical devices. The most commonly used approach to control tissue responses, particularly inflammation, to more complex devices has been the systemic administration of anti-inflammatory agents, such as corticosteroids. Such systemic administration can result in side effects, however, such as generalized immunosupression, bloating, and psychiatric problems, especially over the long term.

Similar to biofouling seen in biomedical devices, surface adhesion of proteins and other biological material present in marine environments can trigger the beginning of marine biofouling by creating an attractive environment for the growth of colonies of bacteria, algae, spores, fungi, protozoa, barnacles, and the like. The attachment of such organisms to ships' hulls can dramatically increase drag and, therefore, fuel consumption. For example, biofouling can increase fuel consumption by as much as 30%. See Brady, R. F., Jr.; J. Coat. Technol., 72, 44-56 (2000). The presence of the organisms or of the chemicals that they produce also can enhance corrosion by seawater, affecting the structural integrity of numerous man-made marine structures, such as buoys, piers, jetties, and offshore oil and gas platforms.

Conventional coatings used in marine industries to reduce biofouling often contain organo-tins, such as tri-n-butyl tin (TBT). Contamination of seaports and shipping lanes with organo-tins, however, has caused concern. The presence of organo-tins in the marine environment, even at levels in the parts per billion, has toxic effects on marine life other than fouling agents, especially to mollusks, such as oysters and whelks. Many countries have imposed restrictions on the use of organo-tin coatings. Alternative copper oxide based coatings are less toxic, but typically are useful for a shorter period of time, often for no longer than two years. See U.S. Pat. No. 5,449,553 to Griffith, J. R.

Much interest in the development of new non-toxic coatings for preventing biofouling has been directed toward polymeric films and materials with low free surface energies, which are non-adhesive toward proteinaceous materials. One class of polymers that has received a great deal of attention in this regard has been silicones. For example, U.S. Pat. No. 5,449,553 to Griffith, J. R. discloses a nontoxic antifouling coating on a substrate, such as a ship's hull, which includes a two-component system of a release layer and a bonding layer, wherein the release layer is bonded to the bonding layer, which in turn in bonded to the substrate. Both layers contain organopolysiloxanes. Over time, however, the siloxane bonds in such polymers can be hydrolyzed in aqueous environments, causing such coatings to break down. See Bullock, S. et al., J. Colloid Interfac. Sci., 210, 18-36 (1999).

Thus, there exists a need in the art to identify new materials for use in fouling-resistant coatings and materials. In particular, there is a need for new fouling-resistant coatings and materials that are both durable and useful for coating or forming highly complex structures.

### SUMMARY

The presently disclosed subject matter describes acid-derivatized perfluoropolyether (PFPE) materials and their use as coatings, sealants, and flexible fillers, and as structural parts for a variety of devices and apparatuses for medical applications, in particular for use in applications where silicone typically has been used. Further, the presently disclosed acid-derivatized PFPE materials can be used as coatings, sealants, flexible fillers, and structural parts for vessels, structures, and machinery that is exposed to marine environments. The use of the presently disclosed acid-derivatized PFPE materials for such applications is advantageous in that the presently disclosed acid-derivatized PFPE materials are resistant to fouling. More particularly, the presently disclosed acid-derivatized PFPE materials can inhibit protein adsorption on a surface, which can lead to biofouling of the surface.

The presently disclosed acid-derivatized PFPE materials also resist swelling or shrinking when contacted with solvents, including organic solvents. Further, the surface energy of the presently disclosed acid-derivatized PFPE material is low, which allows the acid-derivatized PFPE material to be used in applications where low adhesion and/or high lubricity is desired. Moreover, the presently disclosed acid-derivatized PFPE material is oxygen permeable, but is impermeable to potentially harmful microorganisms, such as bacteria.

Thus, in some embodiments, the presently disclosed subject matter provides a method of coating a surface with an acid-derivatized perfluoropolyether (PFPE) material, the method comprising coating a substrate with a liquid mixture containing a functionalized cross-linkable perfluoropolyether (PFPE) and an acid or an acid precursor on the substrate; and curing the mixture to form a cross-linked PFPE material. In some embodiments the method comprises treating the cross-linked PFPE material to convert the acid precursor groups into acid groups.

In some embodiments, the presently disclosed subject matter provides a method of coating a surface with an acid-derivatized perfluoropolyether (PFPE) material, the method comprising coating a substrate with a liquid mixture containing a functionalized perfluoropolyether (PFPE) and an acid; and curing the mixture to form an acid-derivatized PFPE material.

In some embodiments, the presently disclosed subject matter provides a device, such as an implantable medical device, or a structure, such as an orthopedic apparatus, which is fabricated either in whole or in part from an acid-derivatized PFPE material.

Accordingly, in some embodiments, the presently disclosed subject matter provides a medical device that can have a portion that is formed from an acid-derivatized PFPE material, or is coated with an acid-derivatized PFPE material. Exemplary medical devices include, but are not limited to, adaptors, applicators, aspirators, bandages, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, pacemakers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

In some embodiments, an acid-derivatized PFPE material can be used to hermetically seal implantable electronic devices. For example, a housing of an implantable electronic device that contains one or more electronic components therein can be sealed with an acid-derivatized PFPE material to deter the ingress of moisture and foreign material into the housing when the electronic device is implanted within the body of a subject.

In some embodiments, the presently disclosed subject matter provides an intraluminal prosthesis (e.g., a stent) having tubular body portions comprising oxygen permeable, microbe impermeable acid-derivatized PFPE material. In some embodiments, one or more pharmacological agents can be elutably trapped within the acid-derivatized PFPE material (or otherwise attached to the acid-derivatized PFPE material) of the presently disclosed intraluminal prosthesis. The PFPE material can be configured to allow the one or more pharmacological agents to elute therefrom (e.g., at a predetermined rate) when the intraluminal prosthesis is deployed within a body of a subject. In some embodiments, a pharmacological agent can be homogeneously distributed on the tubular body portion of the intraluminal prosthesis. In some embodiments, a pharmacological agent can be heterogeneously distributed on the tubular body portion of the intraluminal prosthesis.

In some embodiments, the presently disclosed subject matter provides a method of implanting an arterio-venous (AV) shunt within the body of a subject, the method comprising implanting a tubular body comprising oxygen permeable, microbe impermeable acid-derivatized PFPE material within the body of a subject, and then connecting the tubular body to blood vessels in the body to form a shunt therebetween. The tubular body can include one or more pharmacological agents and the acid-derivatized PFPE material of the tubular body can be configured to allow the one or more pharmacological agents to elute therefrom when the shunt is deployed within a body of a subject.

In some embodiments, the presently disclosed subject matter provides artificial blood vessels for insertion within the body of a subject, wherein the artificial blood vessels comprise oxygen permeable, microbe impermeable acid-derivatized PFPE material. In some embodiments, one or more pharmacological agents can be elutably trapped within the acid-derivatized PFPE material (or otherwise attached to the acid-derivatized PFPE material).

In some embodiments, an orthopedic device or apparatus is provided that can be configured to be implanted within the body of a subject, wherein the orthopedic device or apparatus comprises an outer surface of an oxygen permeable, microbe impermeable acid-derivatized PFPE material. Utilizing the presently disclosed acid-derivatized PFPE material with removable implants of any type is advantageous because tissue in-growth can be minimized, thus making removal of the implant safer and less traumatic.

In some embodiments, the presently disclosed subject matter provides bandages and other wound healing devices (e.g., sutures) that include oxygen permeable, microbe impermeable acid-derivatized PFPE material. Such wound healing bandages and devices can include one or more pharmacological agents for treating damaged tissue.

In some embodiments, the presently disclosed subject matter provides an artificial tissue material for use within the lungs of a patient, the artificial tissue material comprising a membrane of acid-derivatized PFPE material that simulates alveolar action.

In some embodiments, the presently disclosed subject matter provides a material for use within a heart-lung machine, the material comprising a membrane of acid-derivatized PFPE material that enhances gas exchange during artificial respiration.

In some embodiments, the presently disclosed subject matter provides an intraocular implant comprising an oxygen permeable, microbe impermeable acid-derivatized PFPE material.

In some embodiments, the presently disclosed subject matter provides a contact lens comprising an oxygen permeable, microbe impermeable acid-derivatized PFPE material.

In some embodiments, the presently disclosed subject matter provides a cochlear implant comprising an oxygen permeable, microbe impermeable liquid acid-derivatized PFPE material.

In some embodiments, the presently disclosed subject matter provides a biosensor comprising an oxygen permeable, microbe impermeable liquid acid-derivatized PFPE material.

In some embodiments, the presently disclosed subject matter provides a method of producing a fabric, the method comprising coating a fabric with a liquid mixture containing a functionalized cross-linkable perfluoropolyether (PFPE) and an acid precursor; curing the mixture to form a cross-linked PFPE material containing acid precursors; and treating the cross-linked PFPE material to transform the acid precursor groups into acid groups. Exemplary fabrics include, but are not limited to, polytetrafluoroethylene, polyamides, polyesters, polyolefins, and polyurethanes, such as LYCRA® (INVISTA, Wilmington, Del., United States of America). In some embodiments, the fabric comprises a non-woven material.

In each of the embodiments described herein, the curing of the mixture of the cross-linkable functionally-capped PFPE material and the acid precursor can be performed by exposing the mixture to heat, light, such as ultraviolet radiation, or other radiation (e.g., microwave radiation and the like). In some embodiments, initiators that facilitate curing can be added to the mixture.

Accordingly, it is an object of the presently disclosed subject matter to provide an acid-derivatized PFPE material for use as coatings, sealants, flexible fillers, and structural parts for a variety of devices and apparatuses for medical applications. It is a further object of the presently disclosed subject matter to provide an acid-derivatized PFPE material for use as coatings, sealants, flexible fillers, and structural parts for vessels, structures, and machinery components exposed to marine environments. These and other objects are accomplished in whole or in part by the presently disclosed subject matter.

Objects of the presently disclosed subject matter having been stated hereinabove, other aspects and objects will become evident as the description proceeds when taken in connection with the accompanying Drawings and Examples as best described herein below.

### BRIEF DESCRIPTION OF THE DRAWINGS

FIG. 1 is a bar graph comparing the absorbance at 450 nm of a variety of polymeric films, the absorbance being indicative of protein interaction with the films, which include polystyrene, poly(ethylene glycol) (PEG), methacryloxy- and styrene-functionalized perfluoropolyether (PFPE), poly(dimethylsiloxane) (PDMS), and the acid-derivatized PFPE material of the presently disclosed subject matter.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

### I. Definitions

The term “biocompatible” as used herein refers to a material that, upon contact with a living element, such as a cell or tissue, causes little or no toxicity.

The term “eluting” as used herein refers to the release of a pharmacological agent from a polymeric material. Eluting also can refer to the release of a material from a substrate via diffusional mechanisms or by release from a polymeric material/substrate as a result of the breakdown or erosion of the material/substrate.

The term “erodible” as used herein refers to the ability of a material to maintain its structural integrity for a desired period of time, and thereafter gradually undergoes any of numerous processes, whereby the material substantially loses tensile strength and mass. Examples of such processes include enzymatic and non-enzymatic hydrolysis, oxidation, enzymatically-assisted oxidation, and others, thus including bioresorption, dissolution, and mechanical degradation upon interaction with a physiological environment into components that the patient's tissue can absorb, metabolize, respire, and/or excrete. The terms “erodible” and “degradable” are intended to be used herein interchangeably.

The term “fluoropolymer,” as used herein, has its conventional meaning in the art. See generally Fluoropolymers (L. Wall, Ed. 1972) (Wiley-Interscience Division of John Wiley & Sons); see also Fluorine-Containing Polymers, 7 Encyclopedia of Polymer Science and Engineering 256 (H. Mark et al. Eds., 2d Ed. 1985). The formation of fluoropolymers is described in U.S. Pat. Nos. 5,922,833; 5,863,612; 5,739,223; 5,688,879; and 5,496,901 to DeSimone et al., each of which is incorporated herein by reference in its entirety.

The term “hydrophobic” is used herein to mean not soluble in water.

The term “hydrophilic” is used herein to mean soluble in water.

The terms “polymer” and “polymeric material” are synonymous and are to be broadly construed to include, but not to be limited to, homopolymers, copolymers, terpolymers, and the like.

The term “toxic materials” is intended to include all types of foreign materials, contaminants, chemicals, physical impurities, and the like, without limitation, that can be harmful to a subject.

As used herein, phrases such as “between X and Y” and “between about X and Y” should be interpreted to include X and Y.

As used herein, phrases such as “between about X and Y” mean “between about X and about Y.”

As used herein, phrases such as “from about X to Y” mean “from about X to about Y.”

As used herein, the term “and/or” includes any and all combinations of one or more of the associated listed items.

As used herein, the term “biofouling” refers to the undesirable contamination of a substrate (e.g., an implantable substrate) by a biological agent. The presently claimed subject matter can encompass biofouling of any substrate by any biological agent (in vivo and in vitro). Representative forms of biofouling with respect to biomedical devices include, but are not limited to protein adhesion, platelet adhesion, and microbial adhesion. Representative microbial species include any commonly associated with medical implant biofouling, such as, but not limited to, Escherichia coli, Staphylococcus epidermidis, Staphylococcus aureus, Streptococcus mutans, Bacillus cereus, Rhodobacter sphaeroides, and Pseudomonas aeruginosa.

The term “prosthesis” is used herein in a broad sense to refer to any artificial device used to replace a body part. For example, an intraluminal prosthesis is a device which is implanted in the body of a subject for some therapeutic reason or purpose including, but not limited to, stents, drug delivery devices, and the like.

Representative biomedical devices include medical implants and medical apparatuses. Biomedical devices include, but are not limited to cardiovascular replacements (e.g., catheters, valves, stents), cardiac pacemakers, vascular grafts, contact and intraocular lenses, shunts, meshes, filters, diaphragms, pumps, membranes, drug delivery devices, insulin pumps, monitoring devices such as blood glucose monitoring systems and body temperature sensors, surgical components, dental implants, and orthopedic prosthetics, such as artificial joints. Additional biomedical devices that can be subject to biofouling include chip-based assays, affinity chromatography columns, biomaterials for tissue engineering, capillary channels for electrophoresis, microfluidics, and biomicroelectromechanical devices.

As used herein, the term “medical implant” refers to an implantable material, a prosthesis, an artificial organ, a repair device, an implantable drug delivery system, or a biosensor. The site of implantation can be anywhere in a body of a subject. Representative implants include both intravascular and subcutaneous sensors, as well as structural implants. Thus, additional representative implants include catheters, orthopedic implants, sutures, staples, stents, breast implants, penile implants, other anatomical implants, screws, nails, plates, rods, and prosthesis. Indeed, any implant as would be apparent to one of ordinary skill in the art upon review of the present disclosure falls within the scope of the presently disclosed subject matter.

As used herein, the term “biosensor” refers to any device that detects and transmits information regarding a physiological change or process. For example, a common biosensor is the blood glucose biosensor, which uses an enzyme to break blood glucose down. During the process, electrons are transferred to an electrode and converted into a measure of blood glucose concentration. Biosensors can use many different types of biologically derived materials or biomimetic materials to detect physiological changes and processes, including, but not limited to, tissues, microorganisms, organelles, cell receptors, enzymes, antibodies, and nucleic acids. Biosensors have been designed to convert information gained from biochemical reactions or interactions involving these materials into an electronic signal based on a variety of physiochemical properties, including heat output or absorption (calorimetric sensors), changes in charge distribution (potentiometric sensors), movement of electrons in a redox reaction (amperometric sensors), light output of a reaction or a difference in the light absorbance of the reactants and the products of a reaction (optical sensors), and effects due to the mass of the products or reactants (piezo-electric sensors).

As used herein, the term “medical apparatus” refers to a device used as part of a medical procedure, such as a surgery or any treatment or medical examination of a patient by a health care professional. Representative medical apparatuses include adaptors, applicators, aspirators, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

The term “lumen” is used herein to refer to any inner open space or cavity of a body passageway.

The term “thrombosis” is used herein to refer to the aggregation of platelets to form a dense network of cells or a thrombus (blood clot). The term “thromboresistant” is used herein to refer to a material or implant that is resistant to biofouling caused by platelet adhesion and subsequent thrombus formation in vitro and/or in vivo. In some embodiments, the presently disclosed acid-derivatized PFPE materials can be used as coatings or structural components of, for example, bedside blood gas/electrolyte instruments, vascular blood pressure monitors, implantable catheters, dialysis units, mechanical extracorporeal circulation devices (i.e., artificial blood-oxygenation), and in vivo intravascular sensors.

The term “subject” is used herein to describe both human beings and animals (e.g., mammalian subjects) for medical, veterinary, testing and/or screening purposes.

As used herein, the term “marine biofouling” generally refers to the impairment or degradation of an article as a result of the attachment, growth, and/or activity of a living organism or organisms thereon. Without being bound to any one particular theory, it is believed that the first stages of marine biofouling can involve the accumulation of organic matter on a substrate, wherein the organic matter can include molecules, such as polysaccharides, proteins and protein fragments. The second stage of marine biofouling can involve the adhesion of single cell organisms, such as bacteria and single cell diatoms to the substrate, forming a microbial biofilm. These organisms secrete muco-polysaccharides and corrosive chemicals, which can increase the roughness of the substrate's surface, thereby attracting further organisms. Further stages of marine biofouling can involve the accumulation of more complex organisms, such as algae, spores, fungi, protozoa, barnacles, limpets, seaweed, sea mats, sea squirts, and the like. The substrate at risk of marine biofouling can be any stone, metal, timber, or plastic material exposed to the marine environment. Thus, substrates for marine biofouling can include heat exchange units, ship-board components (e.g., hulls, superstructure, and the like), installations (e.g., pump derricks), docks, and environmentally exposed instruments.

II. Materials

II.A. Liquid Curable PFPE Materials

Perfluoropolyether (PFPE) materials are a unique class of fluoropolymers that are liquids at room temperature, exhibit low surface energy, low glass transition temperatures, low modulus, high gas permeability, high lubricity, and low toxicity with the added feature of being extremely chemically resistant and thermally stable. PFPE materials are particularly advantageous for use in medical applications because PFPE materials are oxygen permeable, but are impermeable to many pathogens, such as bacteria. Additionally, by comprising liquid, pourable precursors that can be photo or thermally cured, PFPE materials offer the possibility for biomimicry through topographical flexibility. The use of liquid curable PFPE materials for medical applications is described in U.S. Patent Application Publication No. 2005/014315 A1 to DeSimone, J. M., and Williams, M. S., the disclosure of which is incorporated herein by reference in its entirety.

The synthesis of PFPE materials is described generally in W. C. Bunvard et al., Macromolecules 32, 8224 (1999), which is incorporated by reference in its entirety. In general, fluoropolyethers are polymeric compounds comprising multiple, sequentially linked, fluorinated aliphatic ether units (e.g., polymers of the formula (RO)ₙR wherein the R groups are the same or different and are linear or branched, saturated or unsaturated C₁-C₄ alkyl; typically linear or branched saturated C₁-C₄ alkyl, with the number of repeats “n” giving the desired molecular weight). Perfluoropolyethers are such polymers in which essentially all of the hydrogens are substituted with fluorine.

Examples of perfluoropolyethers are illustrated below in Table 1 and include perfluoropolymethyl-isopropyl-ethers such as: (i) polymers marketed under the tradename FOMBLIN®; (ii) polymers marketed under the tradename AFLUNOX®, and (iii) polymers marketed under the tradename FOMBLIN Z_DOL®. See, e.g., U.S. Pat. No. 6,582,823, which is incorporated herein by reference in its entirety.

TABLE 1
Representative Commercially Available PFPE Materials

| | | |
|---|---|---|
| KRYTOX ® | DuPont | 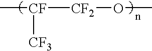 |
| FOMBLIN ® Y | Ausimont | 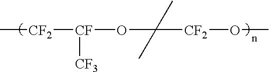 |
| FOMBLIN ® Z | Ausimont | 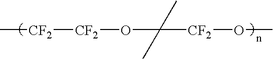 |
| DEMNUM ® | Daikin | 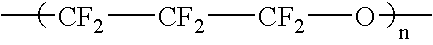 |

The synthesis and photocuring of these materials can be accomplished in a manner similar to that described by Bongiovanni et al., Macromol. Chem. Phys. 198, 1893 (1997), which is incorporated by reference in its entirety. The reaction involves the methacrylate-functionalization of a commercially available PFPE diol (Mₙ=3,800 g/mol) with isocyanato-ethyl methacrylate. Subsequent photocuring of the material is accomplished by blending it with 1 wt % of a photoinitiator, such as 2,2-dimethoxy-2-phenylacetophenone (DMPA), and exposing it to ultraviolet (UV) radiation (A=365 nm) as illustrated below in Scheme 1.

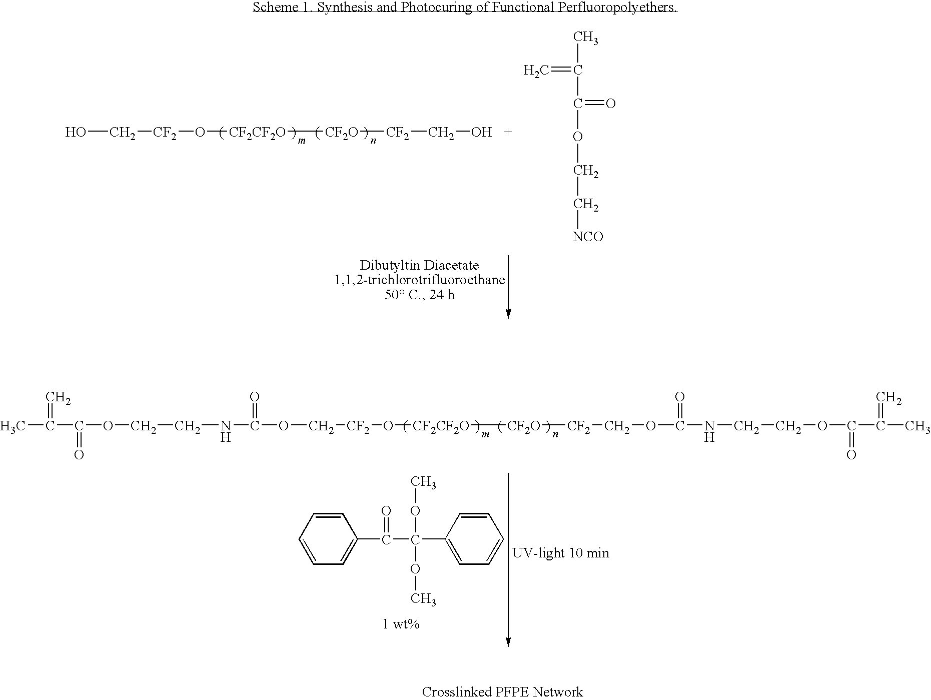

PFPE materials also can be functionalized with various groups, such as epoxy groups, vinyl groups, hydroxyl groups, isocyanate groups, and amino groups, and subsequently cured via various curing mechanisms known to those skilled in the art including, but not limited to, radical, urethane, epoxy, and cationic curing mechanisms. Examples of radical curing include thermal curing with added free radical initiators, such as azo initiators, peroxides, acyl peroxides, and peroxy dicarbonates. Examples of radical curing also include photochemical curing with added photoinitiators (such as 2,2-dimethoxy-2-2-phenylacetophenone) where free radicals are generated photochemically. Epoxy containing PFPE materials can be cured via the addition of amines or by cationic ring-opening methods. Examples of amines useful for curing epoxy containing PFPE materials include 4,4′-diaminodiphenylsulfone. Examples of cationic ring-opening methods for curing epoxy containing PFPE materials include the use of non-ionic or ionic photoacid generators. Useful nonionic photoacid generators include 2,5-dinitrobenzyl tosylate or 2-perfluorohexyl-6-nitro-benzyl tosylate. Useful ionic photoacid generators include diphenyliodium tetraphenyl borate or diphenyliodonium tetra-[3,5-bis(trifluoromethyl)phenyl] borate. Urethane curing mechanisms can include isocyanate reactions with hydroxyl or amine compounds.

PFPE materials according to certain embodiments of the presently disclosed subject matter can be modified and “tuned” to achieve various characteristics and functionalities. For example, reactive monomers can be added to PFPE materials to adjust physical properties including, but not limited to, modulus, wetting, various surface characteristics, and the like.

Reactive monomers that can be added to modify the properties can include styrenics such as styrene, and para-chloromethylstyrene, t-butylstyrene and divinylbenzene; alkyl (meth)acrylates such as butyl acrylate and methyl methacrylate; functional (meth)acrylates such as hydroxyethylmethacrylate, acryloxyethyltrimethylammonium chloride (AETMAC), hydroxyethylacrylate (HEA), cyanoacrylates, fluoroalkyl (meth)acrylates, 2-isocyanatoethyl methacrylate, glycidyl methacrylate, allyl methacrylate and poly(ethylene glycol)diacrylate (PEGdiA); olefins such as norbornene, vinylacetate, 1-vinyl-2-pyrrolidone, and alkylacrylamides.

In addition, various additives can be added to PFPE materials according to embodiments of the present invention including, but not limited to, pharmacological agents, fillers, bioerodible materials, porogens, deoxyribonucleic acid (DNA), oligonucleotides, peptides, growth hormones, and the like. Mechanical fillers that can be added to PFPE materials according to embodiments of the present invention may include, but are not limited to, silica, clay, and other materials of various sizes (e.g., nanoparticles). Additives can be included with PFPE material in various ways including, but not limited to, being chemically attached to PFPE material, being embedded within PFPE material, being dispersed in PFPE material, and the like. The term “attached”, as used herein, encompasses all methods of adding additives to PFPE materials.

In addition, PFPE materials can be tuned to cure as a rigid structure, as a flexible structure, and/or as a partially rigid and partially flexible structure. Moreover, the degree of rigidity and flexibility also can be designed into the PFPE material via additives. Thus, a particular medical device, such as an artificial heart valve, could comprise components fabricated from both a rigid structure, such as a ring, and a flexible structure, such as a leaflet.

In addition, in some embodiments, composite materials having variable layers of rigid and less rigid PFPE materials are used. For example, layers of uniaxially and biaxially oriented materials can be used such that anisotropic properties can be obtained (e.g., flexibility in one direction and strength or rigidity in another direction, and the like).

II.B. Acid-Derivatized PFPE Materials

The presently disclosed acid-derivatized PFPE materials, in some embodiments, can be prepared by mixing a suitably functionalized PFPE and an acid precursor to form a mixture; curing the mixture to form a cross-linked PFPE material comprising acid precursors; and then treating the cross-linked PFPE material comprising acid precursors to convert some or all of the acid precursors into an acid moieties.

In some embodiments, the presently disclosed acid-derivatized PFPE materials can be prepared by mixing a suitably functionalized PFPE and an acid to form a mixture and curing the mixture to form an acid-derivatized PFPE material. In some embodiments, the acid includes, but is not limited to, a sulfonic acid, such as a vinyl sulfonic acid.

In some embodiments, the suitably functionalized PFPE is prepared by reacting an alkenyl halide, such as, for example, 4-vinylbenzyl chloride, with a commercially available PFPE diol. Thus, in some embodiments, the acid-derivatized PFPE materials can be prepared from alkene- or styrene-capped PFPEs. Acid precursors that can be incorporated into such PFPE materials include acid precursors that include alkene or styrene groups that can react with the alkene or styrene groups on the PFPE's via thermal or photo-initiated radical mechanisms.

The acid precursor also includes a masked or protected acid group that can be unmasked or deprotected after the PFPE material has been cured and is miscible with the functionalized PFPE. Suitable acid masking or protecting groups include those that do not interfere or react during the curing process. In some embodiments, the masking or protecting group also adds useful solubility properties to the precursor, so that it can better interact with the functionalized PFPE. For example, suitable acid precursors include esters prepared from acids and fluoro- or perfluorocarbons. In some embodiments, the acid precursor of the presently disclosed subject matter is a vinyl sulfonic acid ester. In some embodiments, the acid precursor is the ester prepared from 4-vinyl benzene sulfonyl chloride and a fluorinated alcohol. In some embodiments, the fluorinated alcohol is 3,3,4,4,5,5,6,6,7,7,8,8,8-tridecafluoro-1-octanol. In some embodiments, the treatment to deprotect or unmask the PFPE to form the final acid-derivatized PFPE material includes an ester hydrolysis step. In some embodiments, the ester hydrolysis step involves base-catalyzed ester hydrolysis.

In addition, the presently disclosed acid-derivatized PFPE materials can be tuned to cure as a rigid structure, as a flexible structure, and/or as a partially rigid and partially flexible structure. Further, the degree of rigidity and flexibility also can be designed into the acid-derivatized PFPE material via additives.

Further, in some embodiments, composite materials having variable layers of rigid and less rigid acid-derivatized PFPE materials are used. For example, layers of uniaxially and biaxially oriented materials can be used such that anisotropic properties can be obtained (e.g., flexibility in one direction and strength or rigidity in another direction, and the like).

In some embodiments, the presently disclosed acid-derivatized PFPE materials can be tuned such that, when cured, the acid-derivatized PFPE material is contiguous, porous, and/or biphasic. Porous or biphasic materials can be achieved by adding other components that will phase separate, such as salts (e.g., sodium chloride); sugars, such as sucrose; water or saline solutions; other polymers, such as polyethylene glycols, poly(vinyl alcohol, or biodegradable polymers, such as polylactides, polyglycolides, polycaprolactone; or added gases or gases that are generated in situ such as through the addition of water to isocyanate compounds, which release CO₂.

In some embodiments, the presently disclosed acid-derivatized PFPE materials can be applied neat, by using a solvent to facilitate the coating process prior to curing, or by plasma deposition. Any solvent that can dissolve the PFPE material is useful. The solvent can reduce the viscosity of the PFPE materials to facilitate the coating process. A lower viscosity can enable the formation of contiguous films or facilitate the formation of thinner films. Exemplary solvents include fluorinated solvents such as FLUORINERT® manufactured by 3M Company (St. Paul, Minn., United States of America).

In some embodiments, curing of the presently disclosed PFPE precursor material(s) in the various applications described herein can be accomplished in a variety of ways including, but not limited to, the use of heat, light and/or other electromagnetic radiation (e.g., microwave, infrared, and the like).

II.C. Pharmacological Agents

In general, pharmacological agents suitable for use with the presently disclosed acid-derivatized PFPE materials include, but are not limited to, drugs and other biologically active materials, and can be intended to perform a variety of functions, including, but not limited to: anti-cancer treatment (e.g., Resan), anti-clotting or anti-platelet formation, the prevention of smooth muscle cell growth, migration, and proliferation within a vessel wall. In some embodiments, pharmacological agents suitable for use with the presently disclosed acid-derivatized PFPE materials include, but are not limited to, antineoplastics, antimitotics, antiinflammatories, antiplatelets, anticoagulants, antifibrins, antithrombins, antiproliferatives, antibiotics, antioxidants, and antiallergic substances, and combinations thereof. Examples of antineoplastics and/or antimitotics include paclitaxel (cytostatic and ant-inflammatory) and its analogs and all compounds in the TAXOL® (Bristol-Myers Squibb Co., Stamford, Conn., United States of America) family of pharmaceuticals, docetaxel (e.g., TAXOTERE® from Aventis S.A., Frankfurt, Germany) methotrexate, azathioprine, vincristine, vinblastine, fluorouracil, doxorubicin hydrochloride (e.g., ADRIAMYCIN® from Pharmacia & Upjohn, Peapack, N.J., United States of America), and mitomycin (e.g., MUTAMYCIN® from Bristol-Myers Squibb Co., Stamford, Conn., United States of America). Examples of antiinflammatories include Sirolimus and analogs thereof (including but not limited to Everolimus and all compounds in the Limus family of pharmaceuticals), glucocorticoids such as dexamethasone, methylprednisolone, hydrocortisone and betamethasone and non-steroidal antiinflammatories such as aspirin, indomethacin and ibuprofen. Examples of antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as ANGIOMAX™ (Biogen, Inc., Cambridge, Mass., United States of America). Examples of cytostatic or antiproliferative agents or proliferation inhibitors include Everolimus, actinomycin D, as well as derivatives and analogs thereof (manufactured by Sigma-Aldrich, Milwaukee, Wis., United States of America; or COSMEGEN® available from Merck & Co., Inc., Whitehouse Station, N.J., United States of America), angiopeptin, angiotensin converting enzyme inhibitors such as captopril (e.g., CAPOTEN® and CAPOZIDE® from Bristol-Myers Squibb Co., Stamford, Conn., United States of America), cilazapril or lisinopril (e.g., Prinivilo and PRINZIDE® from Merck & Co., Inc., Whitehouse Station, N.J., United States of America); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name MEVACOR® from Merck & Co., Inc., Whitehouse Station, N.J., United States of America), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that can be used include alphainterferon, genetically engineered epithelial cells, and dexamethasone.

In some embodiments, pain relief agents also can be added to the presently disclosed acid-derivatized PFPE materials.

III. Applications

In some embodiments, the presently disclosed acid-derivatized PFPE materials can be used in any application where silicone materials have conventionally been used. For example, the presently disclosed acid-derivatized PFPE materials can be used in coatings, sealants, adhesives, structural parts, fillers, implants, and the like. Accordingly, the presently disclosed acid-derivatized PFPE materials can be used in virtually any medical application, product and method. The following sections describe exemplary, nonexclusive embodiments of the presently disclosed subject matter. These embodiments are not intended to encompass the entire scope of embodiments of the presently claimed subject matter.

In some embodiments, these liquid pourable precursors can be cured into any topography which will allow for a biomimetic approach to protein resistant surfaces. Biomimicry is currently an area of focus in the search for a protein resistant surface. Many cellular responses are directed by interaction of cell membrane receptors with protein binding. If a synthetic material can control the body's response to the implant, thus imitating autogenous features, normal cellular functions such as adhesion, accumulation, proliferation, and chemical release can be controlled. The biofouling that occurs at the surface of biomaterials can be reduced using biomimicry in that cell receptors are “confused” and the randomly adsorbed proteins cannot provide an organized signal. This in turn can reduce thrombosis and inflammation around an implanted device, hampering the wound healing process.

III.A. Orthopedic Applications

The presently disclosed acid-derivatized PFPE materials can be used in various orthopedic applications, including orthopedic devices and implants, as well as orthopedic surgical procedures. Embodiments of the presently disclosed subject matter facilitate building and providing new devices and structures for placement within the body of a subject. For example, the presently disclosed acid-derivatized PFPE materials can be used in coating new hip joints. The high wear, high lubricity properties of the presently disclosed acid-derivatized PFPE materials are particularly beneficial for hip joints. The hip joint ball and socket can be coated with the presently disclosed acid-derivatized PFPE material.

The implantable orthopedic apparatus according to embodiments of the presently disclosed subject matter can be artificial or can be cadaver parts refurbished using acid-derivatized PFPE materials. For example, a knee from a cadaver can be refurbished as described hereinabove to improve wear surfaces and to repair damaged areas, and the like.

III.B. Dermatological Applications

The presently disclosed acid-derivatized PFPE materials are particularly advantageous for use in various dermatological applications including, but not limited to, bandages, dressings and wound healing applications, burn care, reconstructive surgery, surgical glue, sutures, and the like. Because acid-derivatized PFPE materials are oxygen permeable and impermeable to potentially harmful microbial organisms, such as bacteria, the tissue underlying a bandage comprising an acid-derivatized PFPE material can receive oxygen while being protected against the ingress of dirt, microbial organisms, pathogens and other forms of contamination and toxicity. Moreover, acid-derivatized PFPE materials are non-toxic. In addition, the oxygen permeability and carrying capacity of acid-derivatized PFPE materials can facilitate the prevention of necrosis in healthy tissue under bandages and dressings or under an area being treated.

In some embodiments, the presently disclosed acid-derivatized PFPE materials can be modified to include adhesive properties so that the acid-derivatized PFPE material can serve the function of a non-toxic, curable liquid bandage for sealing wounds. Exemplary materials that can be added to acid-derivatized PFPE materials to achieve adhesiveness include cyanoacrylates. When cured, the acid-derivatized PFPE material is flexible, yet remains adhered to moving parts such as knees and elbows. In addition, bandages formed from this material provide barriers to infection, can reduce pain to the wearer because of lower surface energy, and can control bleeding better than traditional bandages.

According to embodiments of the presently disclosed subject matter, acid-derivatized PFPE materials can be used in adhesion prevention products for various post-surgical tissue applications. For example, acid-derivatized PFPE material can be applied to post-surgical tissue to prevent other materials and tissue from adhering to the post-surgical tissue. Thus, the presently disclosed acid-derivatized PFPE material can be applied in post-lung lobectomy, hysterectomy, appendectomy, hernia repair or any application where tissue has been injured and connective growth to surrounding tissues or organs is not desired.

III.C. Cardiovascular and Intraluminal Applications

In some embodiments, the presently disclosed acid-derivatized PFPE materials can be used in various cardiovascular applications and in various other intraluminal applications, including devices and methods. Acid-derivatized PFPE materials according to embodiments of the presently disclosed subject matter can be used in blood analysis and treatment devices.

In some embodiments, artificial blood vessels having oxygen permeable, microbe impermeable acid-derivatized PFPE materials can be produced for replacing damaged and/or occluded vessels within the body of a subject. Not only can acid-derivatized PFPE materials serve as conduits for blood flow, but they also can allow for diffusion of oxygen and nutrients through the vessel wall into surrounding tissues thus functioning much like a normal healthy blood vessel to various areas of the body of a subject.

According to embodiments of the presently disclosed subject matter, replacement blood vessels (as well as other cardiovascular vessels) incorporating acid-derivatized PFPE materials can be produced ex vivo for subsequent surgical implantation within the body of a subject.

Embodiments of the presently disclosed subject matter are particularly advantageous regarding replacement of blood vessels. Given their high oxygen carrying ability and permeability, artificial vessels formed from acid-derivatized PFPE materials according to embodiments of the presently disclosed subject matter have highly functional properties with synthetic vasa vasorum characteristics. The presently disclosed acid-derivatized PFPE materials allow diffusion of oxygen through the walls and into surrounding dependent tissues, allow diffusion of sustaining nutrients, diffusion of metabolites. Further, because the presently disclosed acid-derivatized PFPE materials are flexible and compliant, they can mimic vessels mechanically. Moreover, embodiments of the presently disclosed subject matter are particularly suitable for use in heart by-pass surgery and as artificial arterio-venous (AV) shunts. The presently disclosed acid-derivatized PFPE materials also can be used to repair natural or synthetic AV shunts by coating the inside surface of the damaged or worn vessel and curing as previously described.

Acid-derivatized PFPE materials according to embodiments of the presently disclosed subject matter can be used in various intraluminal applications including, but not limited to, stents (and other tissue scaffolding devices), catheters, heart valves, electrical leads associated with rhythm management, balloons and other angioplasty devices, drug delivery devices, and the like. Moreover, the presently disclosed acid-derivatized PFPE materials can be embodied in the material(s) of these devices or in coatings on these devices.

Intraluminal prostheses provided in accordance with embodiments of the presently disclosed subject matter can be employed in sites of the body other than the vasculature including, but not limited to, the biliary tree, the esophagus, the bowels, the tracheo-bronchial tree, the urinary tract, and the like.

Stents are typically used as adjuncts to percutaneous transluminal balloon angioplasty procedures, in the treatment of occluded or partially occluded arteries and other blood vessels. As an example of a balloon angioplasty procedure, a guiding catheter or sheath is percutaneously introduced into the cardiovascular system of a patient through, for example, the femoral arteries and advanced through the vasculature until the distal end of the guiding catheter is positioned at a point proximal to the lesion site. A guidewire and a dilatation catheter having a balloon on the distal end are introduced through the guiding catheter with the guidewire sliding within the dilatation catheter. The guidewire is first advanced out of the guiding catheter into the patient's vasculature and is directed across the arterial lesion. The dilatation catheter is subsequently advanced over the previously advanced guidewire until the dilatation balloon is properly positioned across the arterial lesion. Once in position across the lesion, the expandable balloon is inflated to a predetermined size with a radiopaque liquid at relatively high pressure to radially compress the atherosclerotic plaque of the lesion against the inside of the artery wall and thereby dilate the lumen of the artery. The balloon is then deflated to a small profile so that the dilatation catheter can be withdrawn from the patient's vasculature and blood flow resumed through the dilated artery.

Balloon angioplasty sometimes results in short or long term failure (restenosis). That is, vessels can abruptly close shortly after the procedure or restenosis can occur gradually over a period of months thereafter. To counter restenosis following angioplasty, implantable intraluminal prostheses, commonly referred to as stents, are used to achieve long term vessel patency. A stent functions as scaffolding to structurally support the vessel wall and thereby maintain luminal patency, and are transported to a lesion site by means of a delivery catheter.

Types of stents can include balloon expandable stents, spring-like, self-expandable stents, and thermally expandable stents. Balloon expandable stents are delivered by a dilitation catheter and are plastically deformed by an expandable member, such as an inflation balloon, from a small initial diameter to a larger expanded diameter. Self-expanding stents are formed as spring elements which are radially compressible about a delivery catheter. A compressed self-expanding stent is typically held in the compressed state by a delivery sheath. Upon delivery to a lesion site, the delivery sheath is retracted allowing the stent to expand. Thermally expandable stents are formed from shape memory alloys which have the ability to expand from a small initial diameter to a second larger diameter upon the application of heat to the alloy.

In some embodiments, the presently disclosed acid-derivatized PFPE materials can be used with all of the above-described cardiovascular and intraluminal devices. Thus, the presently disclosed acid-derivatized PFPE materials can be used in the material(s) of these devices and/or can be provided as a coating on these devices.

It can be desirable to provide localized pharmacological treatment of a vessel at the site being supported by a stent or other intraluminal device. Thus, sometimes it is desirable to use a stent both as a support for a lumen wall as a well as a delivery vehicle for one or more pharmacological agents. The presently disclosed acid-derivatized PFPE materials can be configured to carry and release pharmacological agents. Further, the presently disclosed acid-derivatized PFPE materials can be impregnated with pharmacological agents for delivery within a body of a subject. The impregnation of polymer materials is described in commonly assigned U.S. patent application Publication No.: 2004-0098106-A1, which is incorporated herein by reference in its entirety.

In some embodiments, the presently disclosed acid-derivatized PFPE materials can be used in endoluminal sealing processes wherein the interior surfaces of tissue lumens are covered with polymeric material. Acid-derivatized PFPE materials are especially suitable for these procedures because of high lubricity and high permeability to oxygen.

According to embodiments of the presently disclosed subject matter, acid-derivatized PFPE materials can be incorporated into various types of patches used in lung surgical procedures. In some embodiments, preformed patches configured to be attached and secured to lung tissue via conventional methods also can incorporate the presently disclosed acid-derivatized PFPE material.

The use of a patch secured to lung tissue, such as over a wound from tumor removal or a rough surface of the lung, provides a seal to close the wound and prevent air leakage. Additionally, a patch incorporating acid-derivatized PFPE materials can be used in conjunction with sutures and staples to provide additional sealing over the mechanical closures, for example, over the staple or suture line of a lobectomy. The oxygen carrying ability and permeability of the presently disclosed acid-derivatized PFPE materials makes them particularly suitable for use in lung repair. Moreover, because the presently disclosed acid-derivatized PFPE materials can be cured to a flexible state, they are particularly suitable for use as patches for lungs where expansion of a lung requires a flexible and strong bond with a gas-tight seal. In some embodiments, the presently disclosed acid-derivatized PFPE materials can include one or more pharmacological agents that can be configured to elute therefrom, as described hereinabove, when a patch is implanted within a subject's body.

In some embodiments, acid-derivatized PFPE materials can be used in arterio-venous (“AV”) shunts. As known to those skilled in the art, AV shunts are used to join an artery and vein, allowing arterial blood to flow directly into the vein. The presently disclosed acid-derivatized PFPE materials can be used to repair AV shunts or create artificial ones. In some embodiments, the presently disclosed acid-derivatized PFPE materials comprise one or more pharmacological agents that are configured to elute therefrom, as described hereinabove, when a shunt is implanted within a subject's body.

In some embodiments, AV shunts used in dialysis treatment of patients can be replaced and/or repaired using acid-derivatized PFPE materials. AV shunts implanted within dialysis patients periodically require replacement or repair. In some embodiments, existing shunts can be removed and replaced with shunts coated with acid-derivatized PFPE materials.

In some embodiments, the presently disclosed acid-derivatized PFPE material can be used in trachea and esophagus patches. Patches according to embodiments of the presently disclosed subject matter can be effective in preventing or reducing air leakage and/or food leakage from a damaged trachea and esophagus. Preformed patches configured to be attached and secured to trachea/esophagus tissue via conventional methods can include the presently disclosed acid-derivatized PFPE material. In some embodiments, acid-derivatized PFPE materials can include one or more pharmacological agents that are configured to elute therefrom when a patch is implanted within a subject's body.

In some embodiments, because they can enhance gas exchange during respiration, the presently disclosed acid-derivatized PFPE materials can be used as artificial lung material. For example, the presently disclosed acid-derivatized PFPE materials can be used as substitute alveolar membrane material, both for an actual lung and for artificial lung machines and heart-lung machines. As known to those skilled in the art, the alveoli are components within the lung that facilitate oxygen/carbon dioxide exchange and the alveolus is a terminal sacule of an alveolar duct where gases are exchanged during respiration. The high oxygen exchange capacity of the presently disclosed acid-derivatized PFPE materials helps simulate the alveolar action of lung material, including alveoli and alveolus.

III.D. Vision and Hearing Applications

In some embodiments of the presently disclosed subject matter, ocular implants and contact lenses are coated with acid-derivatized PFPE material. It is believed that these devices are advantageous over conventional ocular implants and contact lenses because the acid-derivatized PFPE material is permeable to oxygen and resistant to biofouling. In addition, because of the low surface energy, there is more comfort to the wearer because of lower friction. In addition, the refractive index of acid-derivatized PFPE materials can be tuned (adjusted/precisely controlled) for optimum performance for ocular implants and contact lenses.

In some embodiments, cochlear implants comprising the presently disclosed acid-derivatized PFPE material are advantageous over implants formed from conventional materials. By using the presently disclosed acid-derivatized PFPE material, tissue in-growth can be minimized, thus making removal of the device safer and less traumatic.

III. E. Tissue Treatment

Acid-derivatized PFPE materials can be used for scaffolding for new tissue growth according to embodiments of the presently disclosed subject matter. The high oxygen permeability of acid-derivatized PFPE materials is particularly suitable for promoting tissue growth. Tissue also can be replaced with the presently disclosed acid-derivatized PFPE material.

III.F. Other Devices, Systems and Tools

Various devices, including tools and implants, can incorporate the presently disclosed acid-derivatized PFPE material as described hereinabove. Exemplary devices include tubing, fabrics, filters, balloons, catheters, needles and other surgical tools, clamps and devices. These devices can be made from all types of materials including the presently disclosed acid-derivatized PFPE materials themselves, ceramics, glass, metals, polymers and composites thereof. The presently disclosed acid-derivatized PFPE material can be used as coatings or sealants.

In some embodiments, electronic devices configured to be implanted within the body of a subject are sealed with acid-derivatized PFPE material. For example, a housing containing one or more electronic components therein can be hermetically sealed with the presently disclosed acid-derivatized PFPE material, which prevents the ingress of moisture and bio-fouling into the housing when the electronics device is implanted within the body of a subject.

In some embodiments, individual electronic components such as batteries, capacitors, and the like, that are implanted within the body can be hermetically sealed via acid-derivatized PFPE materials. The presently disclosed acid-derivatized PFPE materials can have high dielectric strength and thus can serve as very good electrical insulators.

In some embodiments, medical tools and devices can be coated or sealed with acid-derivatized PFPE material(s). Any type of medical instrument and device can be coated or sealed with acid-derivatized PFPE material(s) including, but not limited to, instruments and devices used in cosmetic surgery, cardiology, dentistry and oral surgery, dermatology, ENT/otolaryngology, gynecology, laparoscopy, neurosurgery, orthopedics, opthalmology, podiatry, urology, veterinary. The following is a non-exhaustive list of instruments and devices that can be coated or sealed with acid-derivatized PFPE materials as described herein: adaptors, applicators, aspirators, bandages, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, pacemakers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

In some embodiments, natural and synthetic fabrics and clothes can be coated or sealed with acid-derivatized PFPE material(s). In particular, acid-derivatized PFPE material(s) can be used to coat expanded polytetrafluoroethylene (also known as a GORETEX® membrane by W.L. Gore & Associates, Inc., Newark, Del., United States of America) materials and their derivatives and then cured. Other fabrics that can be coated include polyamides, polyesters, polyolefins, LYCRA®, and the like. Fabrics coated with the presently disclosed acid-derivatized PFPE material have a low surface energy, which can alter various performance properties of the fabric. For example, a non-woven fabric of Nylon 6,6 can be coated with an acid-derivatized PFPE material to produce a material having similar surface and barrier properties as a GORETEX® membrane, but at a reduced cost.

III.G. Tools and Systems for Applying, Curing, and Monitoring the Application and Curing of PFPE Materials

In addition to the materials and processes described hereinabove, embodiments of the presently disclosed subject matter include the tools and systems required to deliver or use acid-derivatized PFPE materials in medical devices and tools. Such tools and systems includes catheters; syringes; delivery cartridges for resins, curing agents; heat sources; light sources including directed light sources such as wands, light pipes and lasers and indirect light sources such as wide-area bulbs and arrays. These tools and systems can be used for the use or delivery of acid-derivatized PFPE materials ex situ, such as at a factory or custom manufacturing facility. Techniques that can be used for monitoring or inspecting the delivery or use of acid-derivatized PFPE materials, include, but are not limited to, magnetic resonance imaging, ultrasound imaging, x-ray fluoroscopy, Fourier transform infrared spectroscopy, ultraviolet or visible spectroscopy. The presently disclosed acid-derivatized PFPE materials are non-ferromagnetic materials and, thus, are compatible with MRI. Acid-derivatized PFPE materials also have distinctive IR bands and have a very low optical density in the ultraviolet and visible wavelengths.

III.H. Marine Vessels and Marine Structures

The presently disclosed acid-derivatized PFPE materials can be used as coatings, sealants, flexible fillers, and structural parts for vessels, structures, and machinery exposed to a marine environment. Accordingly, the presently disclosed subject matter provides a method for coating a marine vessel or marine structure with an acid-derivatized perfluoropolyether (PFPE) material, the method comprising providing a substrate comprising a marine vessel or marine structure; disposing a mixture of a functionally-capped PFPE and an acid precursor on the substrate; curing the mixture to form a cross-linked PFPE film comprising acid precursors; and treating the cross-linked PFPE film comprising acid precursors to convert the acid precursors into an acid moiety.

In some embodiments, the substrate is a material selected from the group consisting of plastic, metal, stone, timber, ceramic, and combinations thereof. In some embodiments, the substrate comprises one or more sections of a surface of the group of objects selected from a ship's hull, a pier, a jetty, a dock, a buoy, and a derrick. In some embodiments, the substrate can be affixed to one or more portions of a surface of the group of objects selected from a ship's hull, a pier, a jetty, a dock, a buoy, and a derrick.

In some embodiments, the substrate comprises a panel or a plurality of panels, which then can be applied, e.g., through an appliqué-like technique, to a structure to form, for example, the outer layer of ship's hull or other marine vessel or structure as described immediately hereinabove.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Synthesis of Styrene-Capped PFPE (S-PFPE)

As shown in Scheme 2, styrene linkages were added to both chain ends of a perfluoropolyether diol (PFPE α-, ω-diol) by an interfacial reaction to incorporate crosslinkable functionalities. In a typical synthesis, PFPE α-, ω-diol (30 g, 7.89 mmol), 1,1,1,3,3-pentafluorobutane (30 mL), and tetrabutylammonium hydrogensulfate (1.5 g, 4.42 mmol) were added to a round bottom flask. Potassium hydroxide (15 g, 0.27 mol) was dissolved in deionized water (30 mL) and the aqueous solution was then added to the round bottom flask. After addition of 4-vinylbenzyl chloride (3 mL, 19.2 mmol), the reaction mixture was allowed to stir vigorously at 45° C. for 48 h. The product was passed through a 0.22-μm filter to remove the resulting brown solid. The solution was then extracted by deionized water three times and stirred with carbon black for 1 h to remove any impurities. The mixture was passed through a 0.22-μm filter to remove the carbon black and vacuum dried at room temperature to remove the solvent. The resulting product (S-PFPE) is a clear viscous liquid. 400 MHz ¹H NMR (CDCl₃); 63.82 (2H, —CF₂—CH₂—O), 4.65 (2H, —O—CH₂-φ), 5.25 and 5.80 (vinyl, CH₂═), 6.75 (vinyl, —CH═), 7.30-7.50 (4H, aromatic).

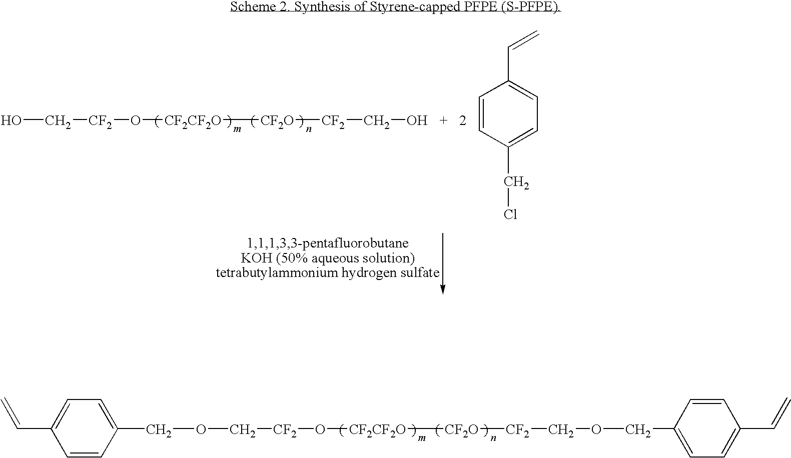

### Example 2

### Synthesis of Styrene Sulfonic Acid Precursor

To make a styrene sulfonic acid precursor that is miscible with S-PFPE, a fluorocarbon tail was added to 4-vinyl benzene sulfonyl chloride. To a round bottom flask, 4-vinyl benzenesulfonyl chloride (7.6 g, 37.5 mmol), 3,3,4,4,5,5,6,6,7,7,8,8,8-tridecafluoro-1-octanol (13.66 g, 37.5 mmol), triethylamine (10 mL), and pyridine (20 mL) were added under argon flow (see Scheme 3). The resulting slurry was stirred at room temperature for 20 h. The reaction mixture was then poured into an ice bath containing excess hydrochloric acid to quench the triethylamine. The aqueous solution was extracted with diethyl ether three times, and the combined ether layer was washed with water, 10% sodium hydroxide solution, and 10% sodium chloride solution sequentially. The ether solution was then dried over MgSO₄ for 1 h. MgSO₄ was then filtered out and diethyl ether was removed by vacuum evaporation. 400 MHz ¹H NMR (CDCl₃); δ2.50 (2H, —CF₂—CH₂—CH₂), 4.30 (2H, CH₂—CH₂—O), 5.50 and 5.90 (vinyl, CH₂═), 6.75 (vinyl, ═CH) 7.55-7.90 (4H, aromatic). 400 MHz ¹⁹F NMR (CDCl₃); −81 (3F, CF₃—), −116 (2F, CF₃—CF₂), −122 to −124 (6F, CF₂—CF₂—CF₂—CF₂—CF₂), −126.5 (2F, CF₂—CH₂).

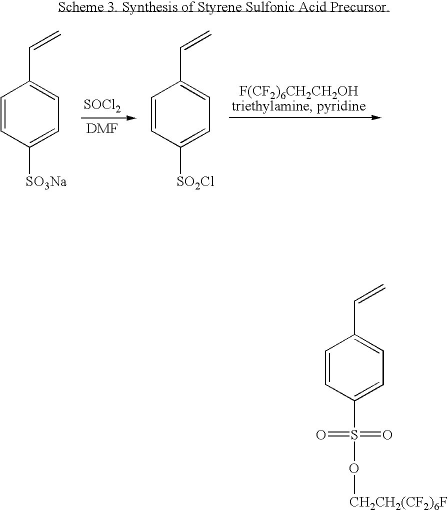

### Example 3

### Preparation of an Acid-Derivatized PFPE Film

As shown in Scheme 4, S-PFPE with 1 wt % photo-initiator (1-hydroxycyclohexyl phenyl ketone) and styrene sulfonate ester were mixed in desired ratios. The mixture was heated above 40° C., e.g., 70° C., to form a homogeneous yellow liquid. The liquid precursor was poured onto a preheated substrate and then chemically crosslinked by irradiation with UV light (λ=365 nm) for 10 min under nitrogen purge.

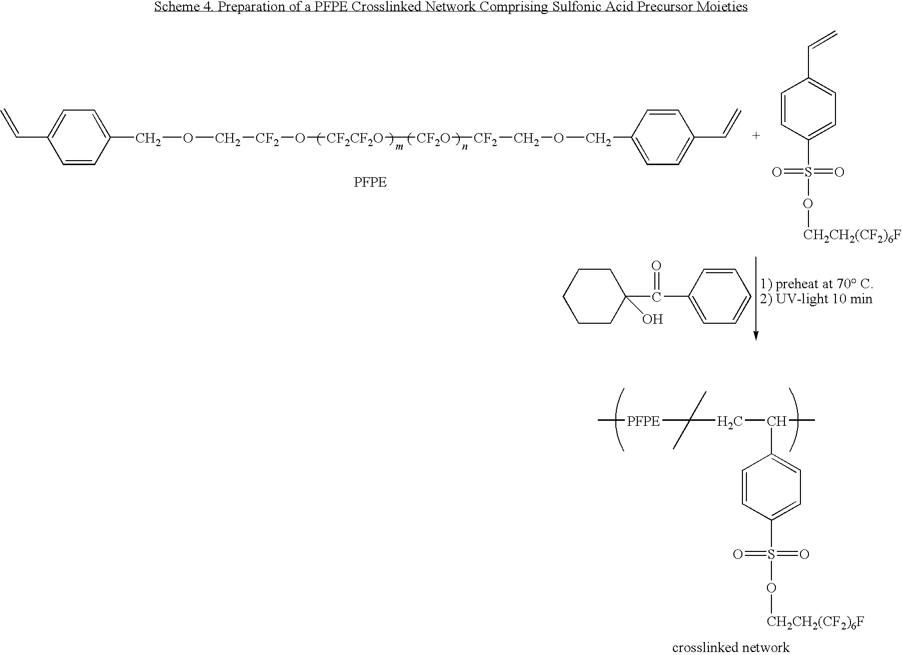

To convert the sulfonate ester group into sulfonic acid, the film was immersed in a mixture of 30% NaOH aqueous solution and methanol (5:6 by volume) overnight and then refluxed for 10 h (see Scheme 5). The film was then rinsed with water and stirred with fresh 20 wt % HCl solution four times over 24 h. The resulting film is in the acid form. Residual HCl was removed by washing with water.

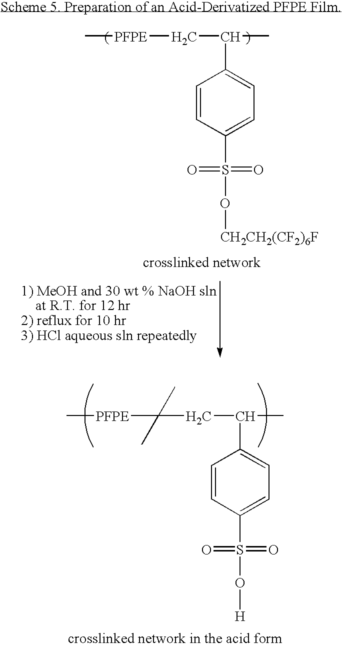

### Example 4

### Protein Adsorption Properties of Acid-Derivatized PFPE Film

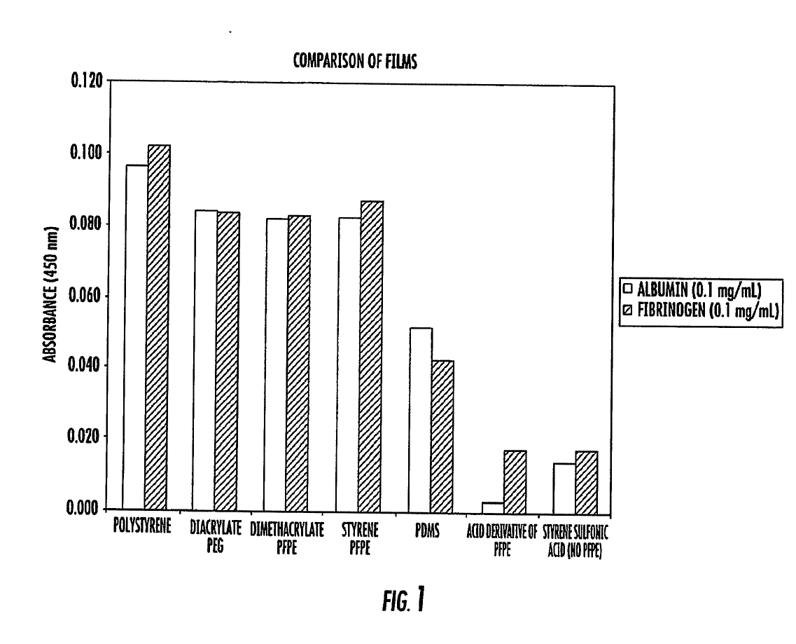

The biofouling resistant properties of the acid-derivatized PFPE film are shown in FIG. 1 and presented in Table 2. The absorbance of albumin or fibrogen proteins interacting with the surface of the film is plotted for various polymeric films. The acid-derivatized PFPE film shows significantly lower absorbance, which is indicative of lower protein adsorption on the polymeric film, compared with films formed from polystyrene, PEG, methacryloxy- and styrene-functionalized PFPE, and PDMS.

TABLE 2
Protein Adsorption on Various Polymeric Films

Absorbance at 450 nm

| | Albumin | Fibrinogen |
|---|---|---|
| Polymer Film | (0.1 mg/mL) | (0.1 mg/mL) |
| Polystyrene | 0.096 | 0.102 |
| Diacrylate PEG | 0.084 | 0.084 |
| Dimethacrylate PFPE | 0.082 | 0.083 |
| Styrene PFPE | 0.083 | 0.087 |
| PDMS | 0.052 | 0.042 |
| Acid Derivative of PFPE | 0.003 | 0.017 |
| Styrene Sulfonic Acid | 0.014 | 0.017 |
| (no PFPE) | | |

### Example 5

### Methods of Testing Protein Adsorption on Films

Unless otherwise noted, all chemicals were purchased from Sigma-Aldrich Co. (St. Louis, Mo., United States of America). 96 well ELISA plates were used for all studies.

Bovine serum albumin (BSA) and canine fibrinogen solutions were prepared by dissolving 0.01 g of protein in 0.67 mL phosphate buffered saline (PBS) to create stock solutions having 15 mg/mL protein concentrations. The solutions were shaken lightly until full dissolution occurred. 100 μL of stock solution was added to a sterile eppendorf tube, and diluted to a concentration of 1.0 mg/mL by adding 1400 μL PBS. To prepare protein solutions having a 0.1 mg/mL protein concentration, 1500 μL of the 1.0 mg/mL solutions were further diluted with an additional 8.5 mL PBS. All solutions were stored at 4° C. and used within a week of preparation.

Solutions of anti-albumin and anti-fibrogen antibodies were prepared by reconstituting the antibodies in 1 mL of HPLC water and then adding PBS to a total volume of 10 mL to achieve a 1:10,000 dilution, as recommended by the manufacturer (Bethyl Laboratories, Inc., Montgomery, Tex., United States of America). The antibody solutions were stored at 4° C.

A positive control study was performed using protein solution (1.0 mg/mL or 0.1 mg/mL) in uncoated wells. All experiments were done in triplicate. 300 mL of protein solution was pipetted into each well. The well plate was capped and the solutions were incubated for 1 h at room temperature. After 1 h, the protein solution was discarded. The wells were rinsed 5 times with 300 μL of PBS and once with 300 μL of PBS-Tween20 solution. 250 μL of the appropriate antibody solution was added to each well and incubated for 10 minutes. The antibody solution was discarded from the wells. The wells were rinsed 5 times with 300 μL PBS. TMB membrane peroxidase substrate was warmed to room temperature and then 100 μL was added to each well and allowed to develop for 30 min. The wells were read using an ELISA plate reader at 450 nm.

A second control study was performed using polyethylene glycol (PEG). PEG-diacrylate was first deinhibited by passage through an alumina column, and then blended with 1 wt % photoinitiator. 10 μL of the PEG was pipetted into each well. The ELISA plate was placed into a UV oven, purged with nitrogen for 10 minutes, and cured for 20 minutes under 365 nm light. 300 μL of protein solution was pipetted into each well. Several wells were used as blanks (i.e., no protein solution was added). The well plate was capped, and the solutions were incubated for 1 h in the hood at room temperature. After 1 h, the protein solution was discarded from the wells using a pipet. The wells were rinsed 5 times with 300 μL of PBS, and 1 time with 300 μL of PBS-Tween20 solution. 250 μL of the appropriate antibody solution was added to each well and incubated for 10 minutes. The antibody solution was discarded from the wells with a pipet. The wells were rinsed 5 times with 300 μL of PBS solution. 100 μL of TMB membrane peroxidase was added to each well and allowed to develop for 30 minutes. The wells were read using an ELISA plate reader at 450 nm.

PFPE-diacrylate and PFPE-distyrene studies were done analogously. PFPE-based materials were synthesized in a manner described hereinabove. 10 μL of the PFPE was pipetted into each well. The ELISA plate is placed into a UV oven, purged with nitrogen with 10 minutes, and cured for 20 minutes under 365 nm light. 300 μL of protein solution was pipetted into each well. Several wells were used as blanks (i.e., no protein solution was added). The well plate was capped, and the solution incubated for 1 h at room temperature. After 1 h, the protein solution was discarded from the wells using a pipet. The wells were rinsed 5 times with 300 μL of PBS, and 1 time with 300 μL of PBS-Tween20 solution. 250 μL of the appropriate antibody was added to each well and incubated for 10 minutes. The antibody solution was discarded from the wells with a pipet. The wells were rinsed 5 times with 300 μL of PBS solution. 100 μL of TMB membrane peroxidase was added to each well and allowed to develop for 30 minutes. The wells were read using an ELISA plate reader at 450 nm.

The acid-derivatized PFPE studies were performed by mixing PFPE-distyrene with an appropriate amount of styrene sulfonic ester monomer in a vial, as shown in scheme 4. Several different degrees of acid-loading were investigated. The vial was heated for 5 minutes to make the two components miscible. 10 μL of the solution was placed into each ELISA well, and cured for 15 minutes under 365 nm light. The polymer was then hydrolyzed to the sulfonic acid by filling the wells with a 6:5 (vol:vol) solution of methanol:30% sodium hydroxide, and allowing it to sit at room temperature for 4-5 h. The plate and solution was then heated to 50-80° C. for 4-5 h. The wells were rinsed with water, then filled with a 2:3 (vol:vol) HCl:water solution and allowed to sit for 1 h. The wells were rinsed and fresh HCl:water solution was added again and incubated for 1 h. This was repeated a third time. 300 μL of the protein solution was pipetted into each well. Several wells were used as blanks. The well plate was capped, and the solution is incubated for 1 h at room temperature. After one hour, the protein solution was discarded from the wells using a pipet. The wells were rinsed 5 times with 300 μL of PBS, and 1 time with 300 μL of PBS-Tween20 solution. 250 μL of the appropriate antibody was added to each well and incubated for 10 minutes. The antibody solution was discarded from the wells with a pipet. The wells were rinsed 5 times with 300 μL of PBS solution. 100 μL of TMB membrane peroxidase was added to each well and allowed to develop for 30 minutes. The wells were read using an ELISA plate reader at 450 nm.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for coating a substrate with an acid-derivatized perfluoropolyether (PFPE) material, the method comprising:
(a) providing a substrate;
(b) disposing a mixture of a functionally-capped PFPE and an acid precursor on the substrate;
(c) curing the mixture to form a cross-linked PFPE film comprising acid precursors; and
(d) treating the cross-linked PFPE film comprising acid precursors to convert the acid precursors into acid moieties.

2. The method of claim 1, wherein the functionally-capped PFPE comprises an alkene-capping group.

3. The method of claim 2, wherein the alkene-capping group comprises a styrene.

4. The method of claim 1, wherein the functionally-capped PFPE comprises a functionally-capped α, ω-PFPE diol.

5. The method of claim 1, wherein the acid precursor comprises an alkene moiety.

6. The method of claim 1, wherein the acid precursor comprises an ester moiety.

7. The method of claim 1, wherein the acid precursor comprises a fluorinated moiety.

8. The method of claim 1, wherein the acid precursor comprises a precursor of a sulfonic acid moiety.

9. The method of claim 8, wherein the acid precursor has the structure:

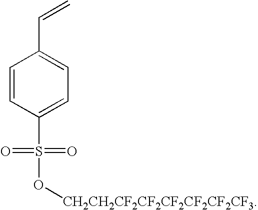

10. The method of claim 1, comprising adding a photoinitiator to the mixture of the functionally-capped PFPE and the acid precursor.

11. The method of claim 10, wherein the photoinitiator comprises 1-hydroxycyclohexyl phenyl ketone.

12. The method of claim 1, comprising heating the mixture of the functionally-capped PFPE and the acid precursor.

13. The method of claim 1, comprising heating the substrate before the disposing of the mixture of functionally-capped PFPE and acid precursor thereon.

14. The method of claim 1, wherein the curing comprises irradiating the mixture of functionally-capped PFPE and acid precursor with actinic radiation.

15. The method of claim 14, wherein the actinic radiation comprises ultraviolet radiation.

16. The method of claim 1, wherein the treating of the cross-linked PFPE film to convert the acid precursor moiety to an acid moiety comprises:
(a) contacting the cross-linked PFPE film with a basic solution for a period of time; and
(b) rinsing the crosslinked PFPE film with water.

17. The method of claim 16, wherein the basic solution comprises sodium hydroxide.

18. The method of claim 17, wherein the basic solution further comprises an alkyl alcohol.

19. The method of claim 16, comprising heating the basic solution.

20. The method of claim 16, further comprising treating the rinsed cross-linked PFPE film with an acidic solution.

21. The method of claim 20, wherein the acidic solution comprises hydrochloric acid.

22. The method of claim 20, further comprising washing the crosslinked PFPE film with water.

23. The method of claim 1, wherein the coating of the substrate with an acid-derivatized PFPE material imparts the substrate with a characteristic selected from the group consisting of reduced protein adhesion, reduced immuno-reactivity, reduced platelet adhesion, reduced microbial adhesion, and reduced chemical corrosion.

24. A method for coating one or more surfaces of a biomedical device with an acid-derivatized perfluoropolyether (PFPE) material, the method comprising:
(a) providing a substrate comprising one or more surfaces of a biomedical device that during its intended use comes into contact with one or more of the group consisting of a cell, a protein, a protein-containing biological fluid and combinations thereof;
(b) disposing a mixture of a functionally-capped PFPE and an acid precursor on the substrate;
(c) curing the mixture to form a cross-linked PFPE film comprising acid precursors; and
(d) treating the cross-linked PFPE film comprising acid precursors to convert the acid precursors into acid moieties.

25. The method of claim 24, wherein the substrate comprises one or more surfaces of an implantable medical device.

26. The method of claim 25, wherein the implantable medical device is selected from the group consisting of a prosthesis, an artificial organ, a repair device, an implantable drug delivery system, and a biosensor.

27. The method of claim 24, wherein the biomedical device is not implanted in a subject and is selected from the group consisting of a biosensor, a chip-based assay device, an affinity chromatography column, and a capillary column for electrophoresis.

28. The method of claim 24, wherein the substrate comprises one or more surfaces of the body of a medical apparatus.

29. The medical apparatus of claim 28, wherein the medical apparatus is selected from the group consisting of: adaptors, applicators, aspirators, bands, blades, brushes, burrs, cables and cords, calipers, carvers, cases and containers, catheters, chisels, clamps, clips, condoms, connectors, cups, curettes, cutters, defibrillators, depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, pacemakers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, stents, syringes, tapes, trays, tubes and tubing, tweezers, and wires.

30. A method for coating a fabric with an acid-derivatized perfluoropolyether (PFPE) material, the method comprising:
(a) providing a fabric;
(b) disposing a mixture of a functionally-capped PFPE and an acid precursor on the fabric;
(c) curing the mixture to form a cross-linked PFPE film comprising acid precursors; and
(d) treating the cross-linked PFPE film comprising acid precursors to convert the acid precursors into acid moieties.

31. The method of claim 30, wherein the fabric is selected from the group consisting of a polytetrafluoroethylene, a polyamide, a polyester, a polyolefin, and a polyurethane.

32. The method of claim 31, wherein the fabric comprises a non-woven material.

33. A medical device having one or more surfaces thereof comprising an acid-derivatized PFPE material.

34. The medical device of claim 33, wherein the device is implantable into a subject.

35. The medical device of claim 34, wherein the device is an orthopedic apparatus configured to be implanted within the body of a subject, wherein the apparatus comprises an outer surface of an acid-derivatized PFPE material.

36. The medical device of claim 35, wherein the apparatus comprises layers of uniaxially and biaxially oriented materials.

37. The medical device of claim 34, wherein the device is a surgical suture, one or more of the outer surfaces thereof comprising an acid-derivatized PFPE material.

38. The medical device of claim 34, wherein the device is an artificial blood vessel, one or more of the outer surfaces thereof comprising an acid-derivatized PFPE material.

39. The medical device of claim 38, wherein the acid-derivatized PFPE material comprises one or more pharmacological agents elutably trapped therein.

40. The medical device of claim 34, wherein the device is intraluminal prosthesis having a tubular body portion having one or more of the outer surfaces thereof comprising an acid-derivatized PFPE material.

41. The medical device of claim 40, further comprising a pharmacological agent elutably trapped within the acid-derivatized PFPE material, and wherein the acid-derivatized PFPE material is configured to allow the pharmacological agent to elute therefrom when the intraluminal prosthesis is deployed within a body of a subject.

42. The medical device of claim 41, wherein the acid-derivatized PFPE material is configured to allow the pharmacological agent to elute at a predetermined rate.

43. The medical device of claim 40, wherein a plurality of pharmacological agents are elutably trapped within the acid-derivatized PFPE material.

44. The medical device of claim 43, wherein the plurality of pharmacological agents are homogeneously distributed on the tubular body portion.

45. The medical device of claim 43, wherein the plurality of pharmacological agents are heterogeneously distributed on the tubular body portion.

46. The medical device of claim 40, wherein the tubular body portion comprises a first end, a second end, and a flow passage defined therethrough from the first end to the second end, wherein the body portion is sized for intraluminal placement within a subject passage, and wherein the body portion is expandable from a first, reduced cross-sectional dimension to a second enlarged cross-sectional dimension so that the body portion can be transported intraluminally to a targeted portion of a passage and then expanded to the second enlarged cross-sectional dimension so as to engage and support the targeted portion of the passage.

47. The medical device of claim 40, wherein the intraluminal prosthesis comprises a stent.

48. The medical device of claim 34, wherein the device is an implantable electronic device, comprising: a housing containing one or more electronic components therein; and an acid-derivatized PFPE material forming a hermetic seal around the housing that deters the ingress of moisture into the housing when the electronics device is implanted within the body of a subject.

49. The medical device of claim 34, wherein the device is one of a cardiac pacemaker and an implantable cardioverter-defibrillator.

50. The medical device of claim 34, wherein the device is an intraocular implant.

51. The medical device of claim 34, wherein the device is a cochlear implant.

52. The medical device of claim 34, wherein the medical device is a dental implant.

53. The medical device of claim 34, wherein the medical device is a biosensor.

54. The medical device of claim 53, wherein the biosensor is selected from the group consisting of a calorimetric biosensor, a potentiometric biosensor, an amperometric biosensor, an optical biosensor, and a piezo-electric biosensor.

55. The medical device of claim 53, wherein the biosensor is a blood glucose biosensor.

56. The medical device of claim 33, wherein the medical device comprises a contact lens.

57. The medical device of claim 33, wherein the medical device is a medical apparatus selected from the group consisting of: adaptors, applicators, aspirators, bandages, bands, blades, brushes, burrs, cables and cords, calipers, carvers, cases and containers, catheters, chisels, clamps, clips, condoms, connectors, cups, curettes, cutters, defibrillators depressors, dilators, dissectors, dividers, drills, elevators, excavators, explorers, fasteners, files, fillers, forceps, gauges, gloves, gouges, handles, holders, knives, loops, mallets, markers, mirrors, needles, nippers, patches, picks, pins, plates, pliers, pluggers, probes, punches, pushers, racks, reamers, retainers, retractors, rings, rods, saws, scalpels, scissors, scrapers, screws, separators, spatulas, spoons, spreaders, syringes, tapes, trays, tubes and tubing, tweezers, and wires.

58. A bandage configured to be applied to the body of a subject, wherein one or more parts of an outer surface of the bandage comprises an acid-derivatized PFPE material.

59. The bandage of claim 58, wherein the acid-derivatized PFPE material comprises one or more pharmacological agents elutably trapped therein.

60. A method of preparing an acid-derivatized PFPE membrane, the method comprising:
(a) mixing a functionally-capped PFPE and an acid precursor to form a mixture;
(b) curing the mixture to form a cross-linked PFPE membrane comprising acid precursors; and
(c) treating the cross-linked PFPE membrane comprising acid precursors to convert the acid precursors into acid moieties.

61. The method of claim 60, wherein the functionally-capped PFPE comprises a styrene-capped PFPE.

62. The method of claim 60, wherein the acid precursor comprises a styrene sulfonate ester.

63. An oxygen-permeable, acid-derivatized PFPE membrane prepared according to the method of claim 60.

64. The membrane of claim 63, the membrane for use as an artificial tissue material within the lungs of a patient to simulate alveolar action.

65. The membrane of claim 63 for use within a heart-lung machine to enhance gas exchange during artificial respiration.

66. A method for coating a marine vessel or marine structure with an acid-derivatized perfluoropolyether (PFPE) material, the method comprising:
(a) providing a substrate comprising a marine vessel or marine structure;
(b) disposing a mixture of a functionally-capped PFPE and an acid precursor on the substrate;
(c) curing the mixture to form a cross-linked PFPE film comprising acid precursors; and
(d) treating the cross-linked PFPE film comprising acid precursors to convert the acid precursors into acid moieties.

67. The method of claim 66, wherein the substrate is a material selected from the group consisting of plastic, metal, stone, timber, ceramic, and combinations thereof.

68. The method of claim 66, wherein the substrate comprises one or more sections of a surface of the group of objects selected from a ship's hull, a pier, a jetty, a dock, a buoy, and a derrick.

69. The method of claim 66, wherein the substrate can be affixed to one or more portions of a surface of the group of objects selected from a ship's hull, a pier, a jetty, a dock, a buoy, and a derrick.

70. The method of claim 66, wherein the functionally-capped PFPE is a styrene capped PFPE.

71. The method of claim 66, wherein the acid precursor is an ester of 4-vinyl phenyl sulfonic acid.

72. A method for inhibiting biofouling on a substrate, the method comprising:
(a) providing a substrate;
(b) disposing a mixture of a functionally-capped PFPE and an acid precursor on the substrate;
(c) curing the mixture to form a cross-linked PFPE film comprising acid precursors; and
(d) treating the cross-linked PFPE film comprising acid precursors to convert the acid precursors into acid moieties.

73. A method for inhibiting protein adsorption on a substrate, the method comprising:
(a) providing a substrate;
(b) disposing a mixture of a functionally-capped PFPE and an acid precursor on the substrate;
(c) curing the mixture to form a cross-linked PFPE film comprising acid precursors; and
(d) treating the cross-linked PFPE film comprising acid precursors to convert the acid precursors into acid moieties.

74. A method of preparing a device or structure comprising an acid-derivatized perfluoropolyether (PFPE) material, the method comprising:
(a) providing a mold or template of the device or structure;
(b) disposing a mixture of a functionally-capped PFPE and an acid precursor on or in the mold or template;
(c) curing the mixture to form a cross-linked PFPE material comprising acid precursors; and
(d) treating the cross-linked PFPE material comprising acid precursors to convert the acid precursors into acid moieties.

75. The method of claim 74, comprising removing the cross-linked PFPE material comprising acid precursors from the mold or template before treating the cross-linked PFPE material comprising acid precursors to convert the acid precursors into acid moieties.

76. A medical device comprising an acid-derivatized PFPE material.

77. The medical device of claim 76, wherein the device is implantable into a subject.

78. A method for coating a substrate with an acid-derivatized perfluoropolyether (PFPE) material, the method comprising:
(a) providing a substrate;
(b) disposing a mixture of a functionally-capped PFPE and an acid on the substrate; and
(c) curing the mixture to form a coating of an acid-derivatized PFPE material on the substrate.

79. A method for coating one or more surfaces of a biomedical device with an acid-derivatized perfluoropolyether (PFPE) material, the method comprising:
(a) providing a substrate comprising one or more surfaces of a biomedical device that during its intended use comes into contact with one or more of the group consisting of a cell, a protein, a protein-containing biological fluid and combinations thereof;
(b) disposing a mixture of a functionally-capped PFPE and an acid on the substrate; and
(c) curing the mixture to form a coating of an acid-derivatized PFPE material on the one or more surfaces of the biomedical device.

80. A method for coating a fabric with an acid-derivatized perfluoropolyether (PFPE) material, the method comprising:
(a) providing a fabric;
(b) disposing a mixture of a functionally-capped PFPE and an acid on the fabric; and
(c) curing the mixture to form a coating of an acid-derivatized PFPE material on the fabric.

81. A method of preparing an acid-derivatized PFPE membrane, the method comprising:
(a) mixing a functionally-capped PFPE and an acid to form a mixture; and
(b) curing the mixture to form an acid-derivatized PFPE membrane.

82. A method for coating a marine vessel or marine structure with an acid-derivatized perfluoropolyether (PFPE) material, the method comprising:
(a) providing a substrate comprising a marine vessel or marine structure;
(b) disposing a mixture of a functionally-capped PFPE and an acid on the substrate; and
(c) curing the mixture to form a coating of an acid-derivatized PFPE on the marine vessel or marine structure.

83. A method for inhibiting biofouling on a substrate, the method comprising:
(a) providing a substrate;
(b) disposing a mixture of a functionally-capped PFPE and an acid on the substrate; and
(c) curing the mixture to form an acid-derivatized PFPE film on the substrate.

84. A method for inhibiting protein adsorption on a substrate, the method comprising:
(a) providing a substrate;
(b) disposing a mixture of a functionally-capped PFPE and an acid on the substrate; and
(c) curing the mixture to form an acid-derivatized PFPE film on the substrate.

85. A method of preparing a device or structure comprising an acid-derivatized perfluoropolyether (PFPE) material, the method comprising:
(a) providing a mold or template of the device or structure;
(b) disposing a mixture of a functionally-capped PFPE and an acid on or in the mold or template; and
(c) curing the mixture to form a device or structure comprising an acid-derivatized PFPE material.
